(19) United States
(12) Patent Application Publication
(10) Pub. No.: US 2003/0212254 A1
(43) Pub. Date: Nov. 13, 2003

(54) Use of an anion exchange resin (emp-7) as solid support for peptide synthesis and affinity chromatography

(76) Inventors: Clovis Ryuichi Nakaie, Sao Paulo (BR); Danielle Alvez Ianzer, Sao Paulo (BR); Eduardo Maffud Cilli, Sao Paulo (BR); Mauricio Martins Rodrigues, Sao Paulo (BR)

Correspondence Address: MORRISON & FOERSTER LLP, 1650 TYSONS BOULEVARD, SUITE 300, MCLEAN, VA, 22102, US

(21) Appl. No.: 10/168,318

(22) PCT Filed: Dec. 20, 2000
(86) PCT No.: PCT/BR00/00160

(30) Foreign Application Priority Data
Dec. 22, 1999 (FR) PI 9906091-4

(51) Int. Cl. C07K 1/02

(52) U.S. Cl. 530/333

(57) ABSTRACT

The present invention refers to the discovery that the anion exchange resin DEAE-Sephadex A50™ might be also used alternatively as polymeric support for peptide synthesis and affinity chromatography. It was possible to synthesize a model octapeptide (angiotensin II) in this resin using the Fmoc-chemistry and when appropriate “linker groups” are used, free peptide cleaved from the resin can be obtained containing C-terminal extremity in carboxylate or carboxamide forms. By considering the feasibility of synthesizing peptide sequence throughout its structure, the repetitive (NANP)₃ sequence found in the antigenic epitope of the sporozoite form of Plasmodium falciparum involved in the malaria transmission was assembled in the DEAE-Sephadex A50 resin. Column with the synthesized (NANP)₃-containing DEAE-Sephadex A50 resin retained quantitatively antibody for (NANP)₃ sequence which was further removed through usual washing process, thus demonstrating the feasibility of using this anion.

This invention refers to two additional applications discovered for a known type of anion exchange resin. These two uses are related to the possibility of applying this type of anion exchanger support as the starting polymer for peptide synthesis as well as for affinity chromatography. In the latter case the resin would present double chromatographic properties, such as affinity for purification of macromolecules using resin-bound peptide sequence as well as anion exchanger properties given by cationic sites spread throughout the resin matrix.

The chromatographic resin related to this invention is the DEAE-Sephadex A-50™, sold worldwide for decades by the Amershan-Pharmacia Biotech (Upsala, Sweden) as a weak anion exchanger resin for column chromatography. This classification is due to the presence of tertiary diethylamine-ethyl (DEAE) groups in its structure which are characterized by having pKa ranging from 9 to 10 (v.g., Analytical Ion-Exchange Procedures in Chemistry and Biology, Khym, J. X., Prentice Hall, Inc., N.J., USA [1974]). Comparatively, the strong anion exchange resins contain instead, quaternary ammonium groups that do not deprotonate regardless the pH of the media.

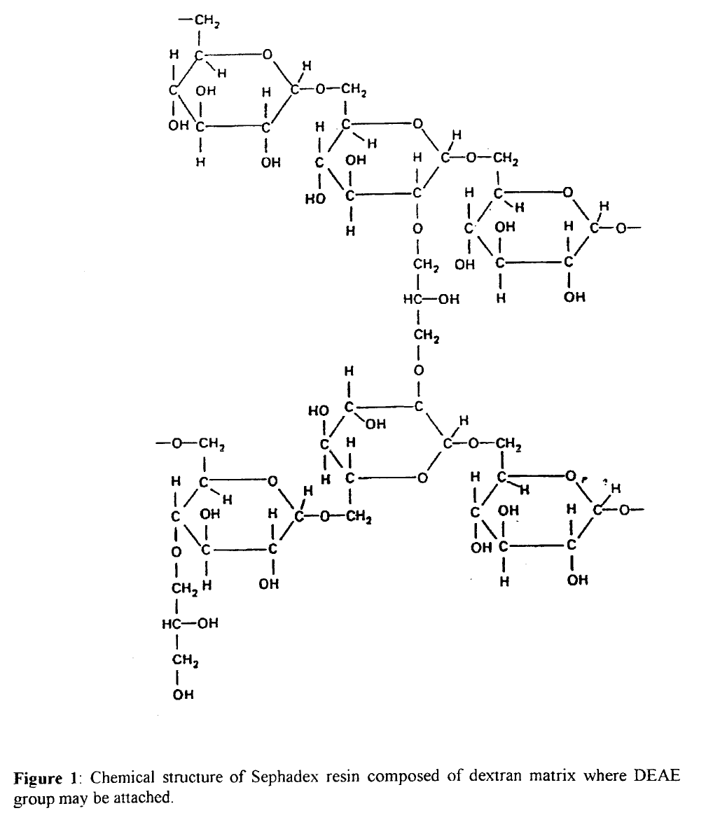

The matrix of the Sephadex-type resin is constituted of a class of carbohydrate—dextran—with variable amount of crosslinkage which defines the exclusion limit of every sub-type of these resins. For the assayed DEAE-Sephadex A50™, this value is around 50 kDa according to the manufacturer catalogue. Sephadex is a type of granular resin with bead diameters varying usually from 50 to 150 μm in the dry form and presents a good swelling either in aqueous or polar organic solvents. FIG. 1 shows the structure of its matrix composed of dextran monomers and may contain different groups such as DEAE.

The idea of testing this kind of anion exchange resin for peptide assembly through the solid phase method (v.g. The Peptides: Analysis, Synthesis and Biology, vol. 2, Academic Press, N.Y., [1980]) resulted from the assumption that during the synthesis of these tertiary amine groups (DEAE)-containing resin, it was plausible the generation of secondary or primary amine groups. If it was true, these more reactive amine groups could serve as attaching site for peptide chain growth through solid phase peptide synthesis. In this case, DEAE-Sephadex A50 would be considered the first resin containing ion exchange sites in dextran-type structure used successfully for peptide synthesis.

The second innovative application of the DEAE-Sephadex™ is simply a consequence of its potential to work as solid support for peptide synthesis. A desired peptide would be assembled in its structure and the composite peptide-resin obtained would serve for purification of macromolecules depending upon the affinity between them and the resin-bound peptide sequence. And if desired, this peptide-resin would function simultaneously as anion exchange and affinity resin for column chromatography.

Thus, the initial experiment with DEAE-Sephadex A50™ was designed to demonstrate, despite the presence of DEAE, groups in its structure, the feasibility of its use for peptide synthesis. The vasoactive angiotensin II (AII) which presents a great variety of physiological functions in the animal organism was selected as peptide model (DRVYIHPF) for this synthesis assay. Besides the doubt concerning the feasibility in assembling peptide chain in its structure, there were also other concernings for instance, the cleavage yield of peptide from the resin. Moreover, due to the fact that DEAE-Sephadex A50 is an aminated resin, the possibility in cleaving peptide will probably generate sequences containing α-carboxamide extremities as usually occurs when aminated resins are used (v.g Peptides 2, 45-50, [1981]). However, if the peptide-resin linkage is stable towards normal cleavage methods there are alternative chemical strategies to remove the synthesized peptide from the resin. These approaches involved the insertion of appropriate chemical groups (“linkers”) between the peptide and the resin which will allow the peptide removal with the C-terminal group as carboxylate or carboxamide forms. Otherwise if the peptide is not cleaved from the resin, it can function as support for affinity chromatography.

Regarding the experiment designed to confirm that DEAE-Sephadex A50 can be applied as affinity resin, it was based initially upon the synthesis of the repetitive [(NANP)₃] sequence, known as antigenic epitope found in the sporozoite form of Plasmodium falciparum (malaria transmission—v.g. Science 225, 593 [1984]). After the peptide synthesis in the resin, the affinity property of the [(NANP)₃]-containing DEAE-Sephadex A50 might be tested against specific antibody generated against this dodecapeptide sequence. In quantitative terms, this assay would be carried out indirectly by calculating the inhibition induced by the addition of the synthesized peptide-resin upon the interaction between the specific antibody for (NANP)₃ segment and a recombinant protein containing this repetitive peptide sequence. As control, this immunological affinity assay was also performed separately with the (NANP)₃-peptide and with DEAE-Sephadex A50 for comparison with the peptidil-Sephadex A50 in this inhibition test.

The results demonstrated a significant decrease in the inhibition of antibody and the recombinant protein interaction when the peptide-resin was added to the reacting medium. This finding confirmed the affinity capacity of this peptide-containing anion exchange resin as it retained in its structure, large molecule such as antibody molecules of (NANP)₃. Besides this type of experiment which confirmed the affinity property of peptide-DEAE-Sephadex A50 complex, additional assays were also carried out to check some of its chromatographic characteristics. It was noticed that antibody molecules were fully retained in the peptide-resin but quantitatively removed from the column when conventional chromatographic washing procedures were applied. This final experiment clearly indicated the feasibility in using peptide-DEAE Sephadex A50 as polymer for liquid affinity chromatography. The following items detail these experiments carried out to demonstrate these unusual properties of the DEAE Sephadex A-type commercial resins.

a) DEAE-Sephadex A50 as Resin for Solid Phase Peptide Synthesis.

The [(9-fluorenylmethyloxy-carbonyl)-N^{α}-temporary protecting group (Fmoc)-strategy (v.g Int. J. Peptide Protein Res. 35, 161-214, [1990]) was tested for peptide synthesis. This method that removes this Fmoc group in basic conditions (20% piperidine in dimethylformamide [DMF] for about 20 min) seemed us more appropriated for AII synthesis. Qualitative amine group detection with the ninhydrin procedure (v.g. Anal. Biochem. 34, 595-598 [1970]) indicated the presence, as formerly assumed, of primary and/or secondary amine groups in DEAE-Sephadex A50 structure, necessary for peptide chain growth (light blue color in the ninhydrin test). As the correct amount of these amine groups in the resin was still unknown, 2 mmol of the first amino acid of the AII sequence (Fmoc-Phe) was used for coupling reaction. After 2 h-coupling, the ninhydrin test was negative indicating complete incorporation of the C-terminal residue.

After acid hydrolysis and Phe determination of the resin through amino acid analysis, a 0,35 mmol/g of primary/secondary amine group substitution degree was determined for the DEAE-Sephadex A50 resin. Based in this value, a 3-fold excess of coupling reagents (Fmoc-amino acid; dicyclohexylcarbodiimide and hydroxybenzotniazole) were used for coupling of the remaining amino acid residues of the sequence and using DMF as solvent for the coupling step. No special difficulties were found along the synthesis and the physical integrity of the resin bead seemed to be maintained. The peptide cleavage from the resin was initially tested using the trifluoroacetic acid (TFA)-containing solution (reagent K) (v.g Int. J. Peptide Protein Res. 35, 161-214, [1990]) but no peptide removal from the resin was observed. This result suggests that the peptide linkage to secondary or primary amine group of this commercial resin is very stable thus presenting the advantage in transforming the peptide-resin, potentially as a support for affinity chromatography. It is possible as the treatment of peptide-resin with reagent K will not cleave the peptide from the resin but all side chain protecting groups of the peptide necessary for its synthesis [All, tert-butyl for Asp, Tyr, Pmc (2,2,5,7,8-pentamethyl-croman-6-sulfonyl) for Arg and trityl for His]. Thus one may have a peptide sequence bound to the DEAE-Sephadex A50 structure free from any temporary side chain protecting groups and being potentially available for affinity interaction with corresponding macromolecules.

As the direct removal of peptide chain from the resin is not possible with the reagent K procedure, we decided to assay chemical “linkers” between the peptide chain and the resin structure introduced specifically for facilitating this final cleavage step with reagent K.

For instance, if the desired peptide contains carboxamide function at the C-terminal position, amongst several chemical groups already known, there is the p-{RS)-[9H-fluore-9-yl)-methoxyformamide]-2,4-dimethoxybenzyl)-phenolacetic acid (FMDF) (v.g. Tetrahedron Lett. 30, 4645 [1989]). Otherwise, if a free carboxylate group-containing sequence is to be synthesized, the 4-hydroxymethylphenoxy-acetic acid (HMPA) (v.g. Int. J. Peptide Protein Res. 20, 451455 [1982]) might be used.

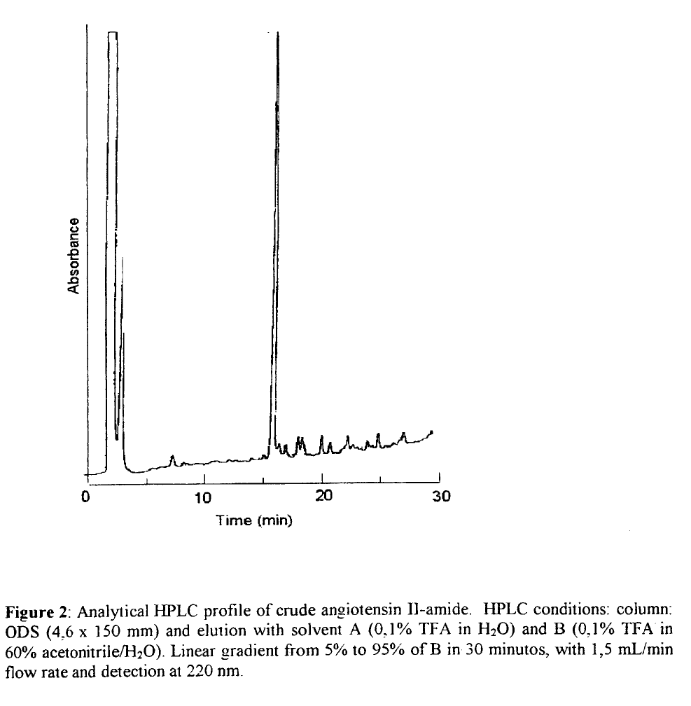

The FMDF linker was used for AII synthesis with DEAE-Sephadex A50 resin and the synthesized peptide-linker-resin complex was treated with reagent K and after precipitation with ethyl ether, the peptide was removed from the resin with 5% acetic acid solution. A white and amorphous powder was isolated after lyophylization of the acid extract. The homogeneity and correct composition of the crude peptide were confirmed by analytical methods such as mass spectrometry, high performance liquid chromatography (HPLC) and amino acid analysis The purity of the crude peptide was rather similar to that observed when conventional resins are used and the corresponding HPLC profile is represented in FIG. 2.

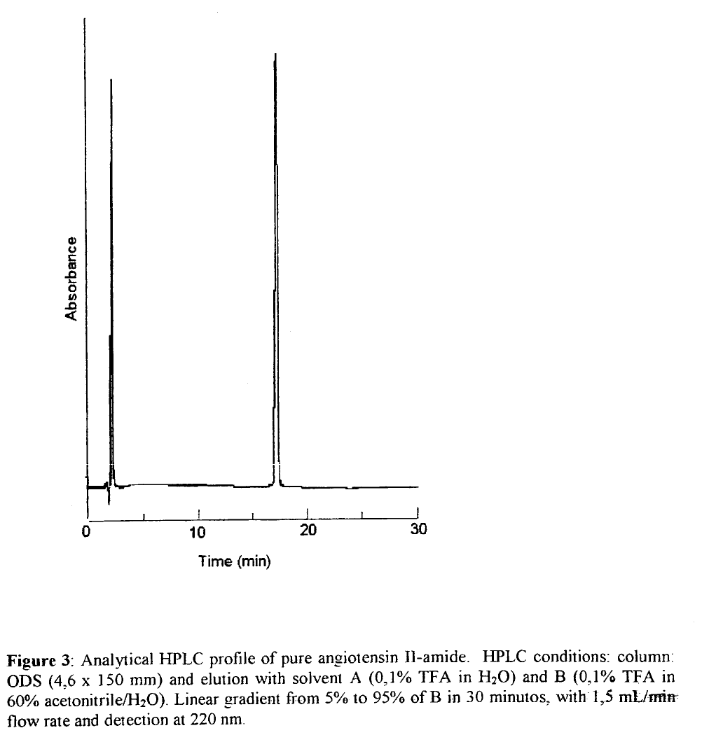

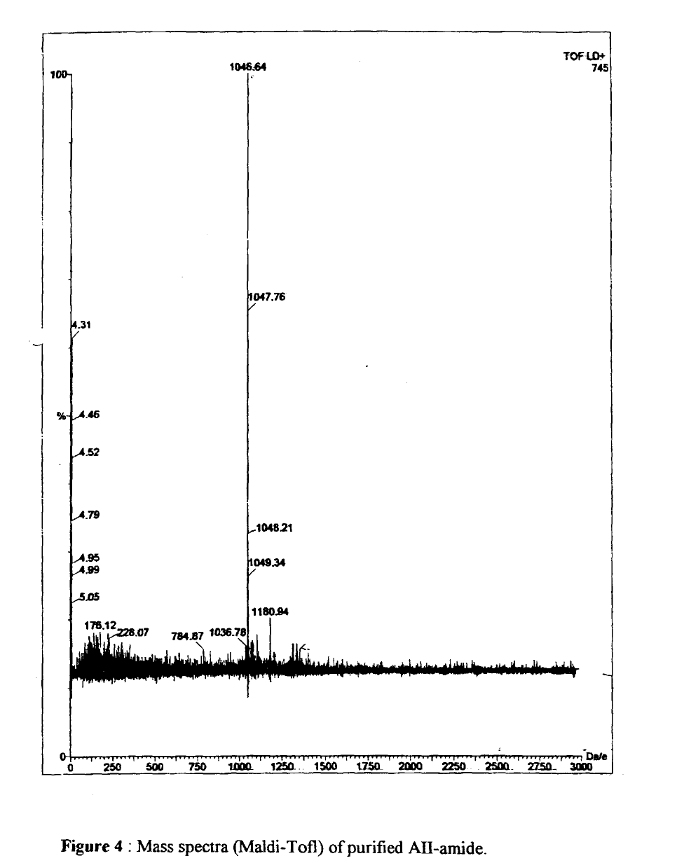

After purification in preparative HPLC (C₁₈ column with pore size of 300 Å, 5 μm of particle size, 4,6×150 mm) and with linear gradient of water/0, 1% TFA (solution A)—water/acetonitrile/0, 1% TFA (solution B), a pure peptide with a final synthesis yield of 63% was obtained. HPLC profile and mass spectra (matrix assisted laser desorption ionization method-Maldi-Tofl) of purified peptide are shown in FIGS. 3 and 4, respectively. These results confirmed that DEAE-Sephadex A50 is the first anion exchange-type resin containing dextran-type matrix that can be used alternatively as solid support for peptide synthesis.

b) Affinity Chromatography Assay with Peptidil-DEAE-Sephadex A50

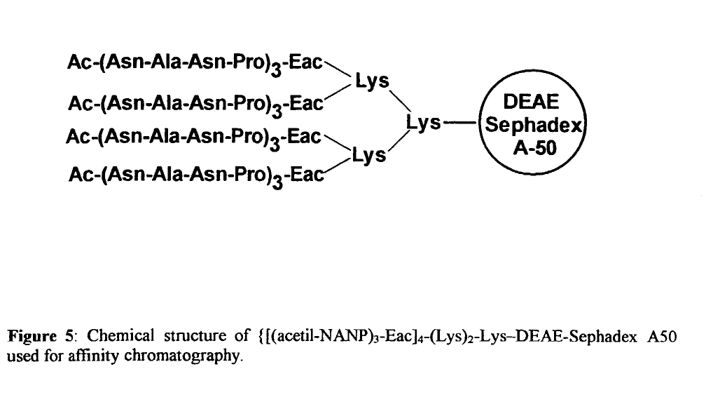

This experiment involved initially the synthesis in DEAE-Sephadex A50 resin, of the repetitive (NANP)₃ sequence, already mentioned as antigenic epitope found in the sporozoite form of Plasmodium falciparum. We decided to acetylate the peptide N-terminal moiety in order to avoid any ion exchange effect during the affinity chromatogram. Moreover to increase the amount of peptide chain in the resin and expecting also for improving the binding of antibody to the peptide sequence, we initiated the synthesis by coupling a lysine residue in the resin (as α,ε-di-Fmoc-Lys derivative). After coupling and Fmoc-removal in basic solution, the two free lysyl-amine groups were again acylated with α,ε-di-Fmoc-Lys, yielding after Fmoc-group removal, a tri-lysyl-branched resin containing four amine groups per molecule available for peptide chain growth. Following a conventional procedure employed in affinity chromatography, a long and inert spacer (ε-aminocaproic acid, Eac) was also coupled in the tri-lysyl-resin core before the peptide chain synthesis aiming to increase the distance from the resin to facilitate the interaction of the peptide segment with the large antibody molecule. The use of this lysyl-branched resin strategy (called MAP or “multiple antigen peptide” approach) has been applied with success to produce antibody for small peptide sequences without the need for binding them in large protein molecules (v.g. Proc. Natl. Acad Sci. it uses, 85, 5409, [1988]). In the present work, the MAP strategy as mentioned, will be used expecting for enhancement of the antibody interaction with the peptide-resin as a consequence of the presence of four (acetyl-NANP)₃ chains in a single molecule linked to the resin. The chemical protocol followed the same applied for AII synthesis but without the FMDF-linker because the objective was to obtain the composite {[(acetil-NANP)₃]₄-Eac₄-(Lys)₂-Lys-DEAE-Sephadex A50} resin for affinity study with the antibody generated from the acetil-(NANP)₃ sequence. The FIG. 5 details the chemical structure of this peptide-resins. Also for affinity investigation, DEAE Sephadex A50 and acetyl-(NANP)₃ were also comparatively evaluated in the binding experiments with the peptide antibody.

In brief, the designed immunological experiment encompassed the following steps: the purified recombinant protein containing the repetitive domain (NANP)ₙ of the circunsporozoite form of Plasmodium falciparum (v.g. Cell, 70, 1021-1033, [1992]) was added to Elisa's plate (200 ng/well) and maintained overnight at room temperature. After 3 washes with phosphate buffer containing 0,05% Tween-20, the plates were treated at 37° C. with PBS containing 5% disnatured milk and 1% serum bovine albumin. To calculate the degree of interaction of this recombinant protein with its antibody, the plates were treated with a specific monoclonal antibody for the (NANP)₃ sequence obtained according to the reference in Science 228, 1436-1440 (1985), in a final concentration of 20 ng/ml. After 2 h at room temperature, the unbound antibodies were washed with PBS-Tween and light and heavy anti-IgG of rat, conjugated to peroxidase and diluted to 1:4000 was added to each of Elisa's plate wells. After 1 h of incubation at room temperature, the excess of labeled antibody was removed during the washings and the calorimetric reaction with o-phenilenediamine was developed and read at 492 nm. As this compound reacts with anti-IgG-peroxidase bound to monoclonal antibody molecules which in turn, interact with the recombinant protein, this method allows the determination of binding degree between antibody and its respective recombinant protein.

The following step involved more directly the estimation-of the affinity degree between the peptide (NANP)₃ bound to DEAE-Sephadex resin and its monoclonal antibody. This estimation (in percentage) was based on the inhibition induced by the peptide-resin against monoclonal antibody and recombinant protein interaction. The inhibition assay for the antibody and free (NANP)₃ or bound to DEAE Sephadex was carried out according to the following procedure:

100 μl of PBS were added to 2000 μg/ml to 3,9 μg/ml of free peptide (acetyl-NANP)₃ or of {[(acetil-NANP)₃]₄-Eac₄-(Lys)₂-Lys-DEAE-Sephadex A50}resin. The peptide-free resin (DEAE-Sephadex) was also solvated in the same manner and in equivalent amount to the peptide-resin control. Next, 100 μl of solution containing 40 ng/ml of mouse monoclonal antibody specific for (NANP)₃, diluted in PBS containing 10% dehydrated and disnaturated milk and 2% BSA was added to this peptide or peptide-resin containing solutions. After 2 h incubation at room temperature with agitation, 60 μl of each sample was transferred to Elisa's plate wells containing recombinant protein. The absorbance of solutions was measured at 492 nm and the percentage of inhibition of this interaction antibody-recombinant protein induced by peptide-resin, resin or peptide was calculated according to the equation:

(1−Abs₄₉₂ with peptide/Abs₄₉₂ without peptide)×100

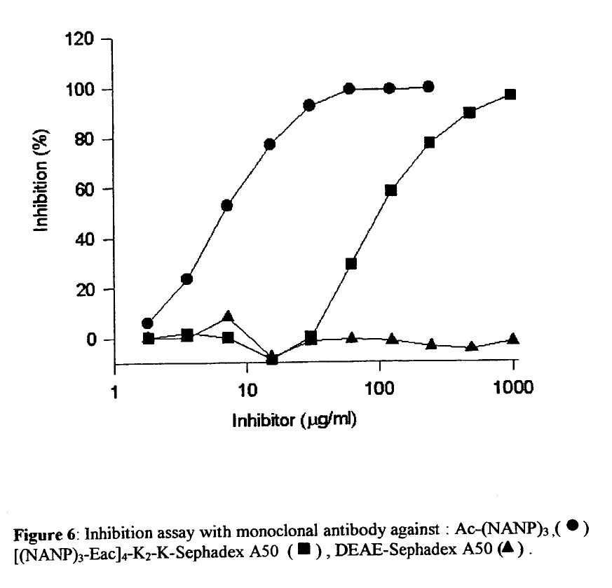

The FIG. 6 compares the result of the affinity amongst {[(acetil-NANP)₃]₄-Eac₄-(Lys)₂-Lys-DEAE-Sephadex A50}, free (acetyl-NANP)₃ or DEAE-Sephadex A50 with the monoclonal antibody. A good inhibition is observed with the peptide-containing resin. As expected the highest inhibition occurred with the free peptide as there is no steric hindrance induced by the resin matrix for interaction with antibody molecule. Otherwise the DEAE-Sephadex A50 resin solely did not showed inhibition thus suggesting that only the presence of positively charged ammonium groups (DEAE) did no affect the results observed with the peptide-resin. In order to better evaluate the potentiality of the mentioned peptide-resin as solid support for affinity chromatography, a known quantity of antibody was added to the resin in a small column. It was observed that the added material was entirely retained in this resin (in neutral pH and for two hours contact). In the subsequent step, the standard acid washing protocol used in affinity chromatography was applied to the resin and the antibody was quantitatively released. The removed antibody maintained its initial interaction capacity towards the recombinant protein thus suggesting that the submission to affinity experiment followed by washings steps did not affected its structural integrity.

These results demonstrated the capacity of this peptide-resin to interact with a large molecule such as an antibody thus confirming that DEAE-Sephadex type-resin, so far used for decades solely as an anion exchange resin, can be transformed alternatively as solid support for affinity chromatography depending on the peptide sequence synthesized throughout its polymeric matrix.

1) “USE OF AN ANION EXCHANGE RESIN (EPM-7) AS SOLID SUPPORT FOR Peptide synthesis and affinity chromatography”, characterized by demonstrating that the commercial anion exchanger resin (DEAE-Sephadex A50™ or similar), by containing primary and/or secondary amine groups in its structure, might be used as starting polymer for peptide synthesis through solid phase method using the base labile (9-fluorenyl-methyloxycarbonyl)-N^{α}-protecting group (Fmoc) and final cleavage of peptide from the resin in highly percentage of trifluoroacetic acid mixture (82,5%)—denominated reagent K—and containing several suppressors for side reactions:

2) “USE OF AN ANION EXCHANGE RESIN (EPM-7) AS SOLID SUPPORT FOR PEPTIDE SYNTHESIS AND AFFINITY CHROMATOGRAPHY”, according with the claim 1, characterized by the need in using a “linker group” such as p-{[RS)-[9H-fluore-9-yl)-methoxyformamide]-2, 4-dimethoxibenzyl)-phenoxyacetic acid (FNDF) so that after the cleavage step, the peptide can be removed from the resin with its C-terminal residue in carboxamide form.

3) “USE OF AN ANION EXCHANGE RESIN (EPM-7) AS SOLID SUPPORT FOR PEPTIDE SYNTHESIS AND AFFINITY CHROMATOGRAPHY”, according with the claim 1, characterized by the need in using a “linker group” such as 4-hydroxymethylphenoxyacetic acid (HMPA) for the synthesis of peptide sequence containing free carboxyl-terminal.

4) “USE OF AN ANION EXCHANGE RESIN (EPM-7) AS SOLID SUPPORT FOR PEPTIDE SYNTHESIS AND AFFINITY CHROMATOGRAPHY”, characterized by demonstrating that DEAE-Sephadex A50 resin, when containing appropriate peptide sequence synthesized throughout its structure may function as affinity support for macromolecules provided that “linker groups” are not used for the synthesis which might induce peptide removal from the resin during reagent K treatment.

5) “USE OF AN ANION EXCHANGE RESIN (EPM-7) AS SOLID SUPPORT FOR PEPTIDE SYNTHESIS AND AFFINITY CHROMATOGRAPHY”, according with the claim 4 above but characterized by the transformation of the DEAE-Sephadex A50 resin in a affinity support through coupling of a pre-purified peptide in its matrix without the need in synthesizing it by the conventional peptide synthesis protocol.

6) “USE OF AN ANION EXCHANGE RESIN (EPM-7) AS SOLID SUPPORT FOR PEPTIDE SYNTHESIS AND AFFINITY CHROMATOGRAPHY”, according with claims 4 and 5 and when containing peptide sequence, the DEAE-Sephadex A50 resin may be used simultaneously or not as affinity and anion exchanger solid support.
